(19) United States
(12) Patent Application Publication
(10) Pub. No.: US 2009/0005842 A1
(43) Pub. Date: Jan. 1, 2009

(54) Cooling System

(75) Inventor: Wen Yan Lin, Taiwan (CN)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: ALPHARMA INC., ANIMAL HEALTH DIVISION, Piscataway, NJ (US)

(21) Appl. No.: 12/279,319

(22) PCT Filed: Oct. 12, 2007
(86) PCT No.: PCT/US07/81274

§ 371 Date: Aug. 13, 2008

Related U.S. Application Data
(60) Provisional application No. 60/851,614, filed on Oct. 13, 2006.

(51) Int. Cl. A61F 7/02; B05D 5/10

(52) U.S. Cl. 607/112; 427/207.1

(57) ABSTRACT

The present invention provides cooling sheets for application to an extremity of a mammal, and methods for making and using such cooling sheets.

### CROSS REFERENCE

This application claims priority to U.S. Provisional Patent Application Ser. No. 60/851,614 filed Oct. 13, 2006, incorporated by reference herein in its entirety.

### BACKGROUND

A 400 to 500 kg horse running at full strength is said to place a load of several tons on each of its legs, and the shock received through the hooves at this time is greater on the lower leg (the portion equivalent to a human's shins and calves) than on the upper leg, and causes fractures, shin splints, tendonitis and other injuries.

Equine bone growth starts from the distal phalanx that comprises the hoof, and ossification proceeds up the leg until the bones for the spine finally mature at age five. Tubular bone periostitis (ie: shin splints) is observed, for example, when young immature horses of less than three first begin training for racing, and is thought to be caused by excess force to the back of tubular bones. Tendonitis occurs in both the front legs and back legs, but occurs more often in the front legs than in the back legs, and is said to be untreatable. Tendonitis occurs when tendon fibers are torn due to the hyperextension of the tendons while running. All of these are severe injuries for horses, particularly race horses, and the first line of prevention or therapy for early inflammation is cooling the affected area using running wager, ice or coolants. However, the running water method is cumbersome while the effect of cooling is reduced in the summer. Cooling the affected area with ice or a coolant is limited to one hour each time, and must be administered three to four times a day, and if left unattended may lead to a risk of frostbite, and as such it must be administered under constant observation.

There remains a need in the art for improved means for equine leg cooling, and cooling of the extremities of mammals in general.

### SUMMARY OF THE INVENTION

In a first aspect, the present invention provides cooling sheets for application to an extremity of a mammal, comprising:

(a) a support body; and

(b) an adhesive layer provided on said support comprising, by weight:
(i) 60-85% of water;
(ii) 5-15% of polyhydric alcohol;
(iii) 0.5%-3% of a crosslinked, acrylic acid-based polymer; and
(iv) 0.1-3% of a compound selected from the group consisting of 5-chloro-2-methyl-4-isothiazolin-3-one; 2-methyl-4-isothiazolin-3-one; 1,2-dibromo-2,4-dicyanobutan; 2-phenoxyethanol and imidurea, or a combination thereof.

In this first aspect, the cooling sheet may further comprise one or more of the following:
(v) 3-8% polyacrylic acid, or salt thereof
(vi) 0.5%-4% anhydrous silica;
(vii) 0.03-0.15% polyvalent metal salt;
(viii) 0.5%-1.5% oxy acid; and
(ix) 1-5% oil based ingredient.

In another aspect, the present invention provides methods for cooling an extremity of a mammal, comprising placing the surface of the cooling sheet adhesive layer of any embodiment of the first aspect of the invention in contact with a mammal's extremity.

In another aspect, the present invention provides methods for making a cooling sheet, comprising

(a) preparing an adhesive layer comprising:
(i) 60-85% by weight of water;
(ii) 5-15% by weight of polyhydric alcohol;
(iii) 0.5%-3% by weight of a crosslinked, acrylic acid-based polymer; and
(iv) 0.1-3% of a compound selected from the group consisting of 5-chloro-2-methyl-4-isothiazolin-3-one; 2-methyl-4-isothiazolin-3-one; 1,2-dibromo-2,4-dicyanobutan; 2-phenoxyethanol and imidurea, or a combination thereof;

(b) applying the adhesive layer to a support body.

In a further embodiment, the present invention provides methods for treating periostitis and/or tendonitis in a mammal, comprising contacting a mammal in need thereof with the cooling sheet of any one of claims 1-19, wherein the contacting is done at an extremity in which the mammal suffers from periostitis and/or tendonitis.

### BRIEF DESCRIPTION OF THE FIGURES

FIG. 1 is an exemplary flow chart for preparing a cooling sheet of the present invention.

FIG. 2 is an exemplary design for the cooling sheet support body.

FIG. 3 is a graphic depiction of the temperature comparisons over time of the cooling sheet wrap versus the mesh over wrap.

FIG. 4 is a graphical depiction of the difference in temperature between the cooling sheet wrap and control.

### DETAILED DESCRIPTION OF THE INVENTION

In a first aspect, the present invention provides cooling sheets for application to an extremity of a mammal, comprising:

(a) a support body; and

(b) an adhesive layer provided on said support comprising, by weight (the percentage is the % by weight with respect to the total weight of the adhesive layer):
(i) 60-85% of water;
(ii) 5-15% of polyhydric alcohol;
(iii) 0.5%-3% of a crosslinked, acrylic acid-based polymer; and
(iv) 0.1-3% of a compound selected from the group consisting of 5-chloro-2-methyl-4-isothiazolin-3-one; 2-methyl-4-isothiazolin-3-one; 1,2-dibromo-2,4-dicyanobutan; 2-phenoxyethanol and imidurea, or a combination thereof.

In this first aspect, the cooling sheet may further comprise one or more of the following:
(v) 3-8% polyacrylic acid, or salt thereof
(vi) 0.5%-4% anhydrous silica;
(vii) 0.03-0.15% polyvalent metal salt;
(viii) 0.5%-1.5% oxy acid; and
(ix) 1-5% oil based ingredient.

The cooling sheets of the present invention may be used to cool an extremity of a mammal, such as an arm, leg, or portion thereof. Suitable mammals for which the cooling sheets can be used include, but are not limited to, horses, humans, dogs, cats, cattle, sheep and other pets and livestock. The cooling sheets may be used over a long period, and may be used to limit and/or treat periostitis, tendonitis, and other indications. The cooling sheets of the present invention provide improved cooling compared to prior transdermal cooling sheets, as disclosed in the examples that follow, and provides optimal adhesiveness and removability for use on a mammalian extremity, such as the lower portion of a leg. The cooling sheets provide prolonged cooling force, and are an easy and safe cooling treatment.

The cooling sheets are adapted to attach to a mammalian extremity, such as the lower portion of the mammal's leg, and can be adequately adhered and released when applied to the mammal's extremity. Adhesive layer adhesion to the mammal's extremity results in evaporation of the moisture in the cooling sheet, and the vaporization heat therefrom causes the skin temperature to rapidly decline immediately after the adhering thereof, even if a cooling stimulant is not included therein, and this cooling effect is maintained for six hours or more.

Since the cooling sheets of the present invention cool the affected area by utilizing vaporization heat produced by the evaporation of the moisture, the cooling effect is greater and lasts longer with larger moisture content in the adhesive layer. Thus, in various embodiments, the adhesive layer contains a water content of 60-85% by weight, 65-75% by weight, 70-75%, 65-85%, 70-85%, 75-85%, or 80-85% by weight.

In various embodiments, the polyhydric alcohol is selected from the group consisting of glycerin and a mixture of glycerin and one or more ingredients selected from the group consisting of propylene glycol, 1,3-butylene glycol, 1,3-Butanediol, ethylene glycol, and dipropylene glycol. In various embodiments, 3-10% by weight of glycerin is used, which strengthens the adhesive force of the adhesive layer without forming too strong an adhesion to the mammal's extremity that can make removal painful, and also provides good moisture retention.

Exemplary crosslinked, acrylic acid-based polymers include, but are not limited to, those disclosed in U.S. Pat. No. 6,433,061, as well as those sold under the trade name CARBOPOL™ by Noveon, Inc. (Cleveland, Ohio; www.noveon.com), including but not limited to Carbopol Ultrez 10, Carbopol 940, Carbopol 934, Carbopol EZ4, Carbopol Aqua SF-1, Carbopol Ultrez 21, etc. In various embodiments, the crosslinked, acrylic acid-based polymer is present in the adhesive layer at between about 1%-3%; 1.5%-3%, 1.5-2.5%, 0.5-2.5%, or 1%-2.5% on a weight:weight basis. The crosslinked, acrylic acid-based polymers is added to the adhesive layer for the purpose of strengthening the gel strength of the adhesive layer, improving the cob webbing caused by the polyacrylate, retaining a large volume of water, and thus helping maintain the cooling effect of the cooling sheet.

The adhesive layer further comprises a compound selected from the group consisting of 5-chloro-2-methyl-4-isothiazolin-3-one; 2-methyl-4-isothiazolin-3-one; 1,2-dibromo-2,4-dicyanobutan; 2-phenoxyethanol and imidurea (also known as imidazolidinyl urea), which provide anti-bacterial activity and may serve to provide additional cooling capacity to the adhesive layer based on an endothermic reaction with water. In one embodiment, Euxyl K 400 (Schulke & Mayr), a preservative containing 1,2-dibromo-2,4-dicyanobutan and 2-phenoxyethanol, can be used. In another embodiment, Katon CG (Rohm & Haas), a preservative containing 5-chloro-2-methyl-4-isothiazolin-3-one and 2-methyl-4-isothiazolin-3-one can be used. In another embodiment, imidurea is used.

The cooling sheet may be provided with an impermeable film covering the surface of the adhesive layer. Such films can be of any suitable size for the purposes of the invention; in one embodiment, this size ranges between about 5-40 cm long and 5-30 cm wide.

In embodiments where the adhesive layer comprises between about 3-8% of a polyacrylic acid (polyacrylate) or salt thereof, any suitable polyacrylate can be used, including but not limited to sodium polyacrylate and/or a polyacrylate neutralizer, or salts thereof, may be used as the polyacrylate. When dissolving polyacrylates and/or the crosslinked, acrylic acid-based polymer in water during the process for preparing the adhesive layer, a liquid can be added, if necessary or desirable, in which the polyacrylates and/or the crosslinked, acrylic acid-based polymer have been dispersed in advance in a polyhydric alcohol to water while stirring. If desirable, a combination of polyhydric alcohols (ie, glycerin, propylene glycol, 1,3-butylene glycol, and/or dipropylene glycol) can be used to disperse the polyacrylates and/or the crosslinked, acrylic acid-based polymer in advance of dissolving in the water component of the adhesive layer.

If present, the oil based ingredient is added to the adhesive layer at between about 1-5% by weight so that it will float to the surface of the adhesive layer after the moisture in this layer has evaporated and weaken the adhesion upon removal. Any suitable oil based ingredient can be used, including but not limited to light liquid paraffin, squalane, mineral oil, liquid polyisobutylene, or other hydrocarbons, olive oil, avocado oil, safflower oil, or other vegetable oils, diisopropyl apidate, isopropyl palmitate, isopropyl myristate, or other esters.

Inclusion in the adhesive layer of anhydrous silica at between about 0.5-4% by weight, or between about 2-3% by weight, helps to improve the gel strength of the adhesive layer and reduce the adhesive force upon removal. Adding anhydrous silica reduces the cob webbing arising in the polyacrylate solution and improves spreading, and also prevents the adhesive layer from seeping out of the support after the sheet shape is formed prior to the shape setting up. In addition, the anhydrous silica retains the polyhydric alcohol and oil based ingredient, and works to prevent these from floating to the surface of the adhesive layer and reducing the adhesive force thereof. Once the moisture has evaporated, the retention of the polyhydric alcohol, which is a plasticizer, hardens to form a polymer film and reduces adhesion, and allows the oil ingredient to float to the surface and reduce the adhesion to make it easy to remove the sheet without ripping out any hairs on the mammal's extremity.

Polyvalent metal salts, when present in the adhesive layer, at between about 0.03-0.15% by weight with respect to the content of the polyacrylate is added to the adhesive layer, and between about 0.5-1.5% by weight of an oxy acid (when present), is activated, and the emitted aluminum bridges the polyacrylate and forms an insolubilized gel. Any suitable polyvalent metal salt can be used, including but not limited to dried aluminum hydroxide gel, aluminum hydroxide, aluminum sulfate, aluminum acetate, aluminum chloride, aluminum glycinal, alum; and salts of zinc, calcium, copper, lead, iron, tin; and combinations thereof.

When present in the adhesive layer, any suitable oxy acid can be used, including but not limited to oxy carbonic acids and/or phosphorous acids (including but not limited to tartaric acid, citric acid, malic acid, or lactic acid), other organic acids, and other inorganic acids that contain a hydroxyl group, or combinations thereof.

The cooling sheets of the present invention, the adhesive layer may further comprise an antiseptic as needed. For example, p-hydroxybenzoic esters, in particular a combination of methyl parahydroxy benzoate and propyl parahydroxy benzoate, are effective as an antiseptic. When including an antiseptic, a solvent that effectively dissolves the p-hydroxybenzoic esters and mixes with water, for example N-methyl-2-pyrrolidone or the like, may be utilized as a solubilizer.

In addition, the adhesive layer may further comprise carboxy vinyl polymer, carboxy methyl cellulose sodium, methyl cellulose, sodium alginate, polyvinyl alcohol, polyvinyl pyrrolidone, methyl vinyl ethyl-maleic anhydride polymer, poly ethylene oxide and other thickening agents, kaolin, ceramic, titanic oxide and other fillers, polyoxy ethylene hardened palm oil, polyoxy ethylene sorbitan fatty acid ester, sorbitan fatty acid ester and other surfactants.

The cooling sheets of the invention may further comprise other components that aid in cooling and/or analgesia, such as peppermint, eucalyptus oil, methyl salicylate, analgesics, and the like.

While the cooling sheet of the present invention posts a sufficient cooling effect with only the adhesive layer, when cold water is sprayed on the support after it has been adhered to the extremity, the support can absorb and retain this water thereby increasing the moisture content regained in the overall cooling sheet and making it possible to maintain the cooling force without worsening the usability of the adhesive layer.

The support body may comprise any water retentive sheet, including but not limited to a non-woven material (stretchable or non-stretchable) containing polyester fibers or and a non-woven material containing one or more fibers selected from highly absorbent fibers. A cooling sheet that utilizes a stretchable non-woven support containing polyester fibers is able to continuously maintain the cooling effect that is the objective of the present invention, but the use of a non-woven support containing highly absorbent fibers is more suitable since a stronger cooling effect can be effectively maintained. Examples of highly absorbent fibers include a highly absorbent resin such as a starch-acrylate graft polymer partial sodium salt bridging substance that has been made into threads, two layer fibers having acryl fibers on the inside and highly absorbent fibers on the outside, threads that have a portion of the cellulose replaced with a hydrophilic group and thereby have increased hydrophilicity, and the like.

In non-woven materials containing only highly absorbent fibers, moisture is absorbed while stored as a non-woven material, and sometimes the adhesive layer will seep into the non-woven material prior to the completion of the bridging of the adhesive layer, but this issue can be avoided by using a non-woven material that contains hydrophilic polyester fibers. Polyester fibers can be processed into highly absorbent fibers by adding a hydrophilic functioning group, and these may be mixed together with ordinary polyester fibers. A non-woven material having such a structure only absorbs and retains water when it comes in contact therewith, and as such it is extremely suitable for use in the manufacturing of the cooling sheet of the present invention. While the manufacturing method for the cooling sheet of the present invention is not specifically limited, manufacturing may be conducted by applying a kneaded adhesive plaster at an even thickness on one side of the support, and then forming a water impermeable film on top thereof. Or, it may be manufactured by evenly covering the water impermeable film with plaster, and then forming a support on top thereof. The thickness of the adhesive layer is between about (ie: +/−10%) 0.8-2.5 mm. The kneading and application methods are not specifically limited, and may utilize any means commonly used in the production of medications.

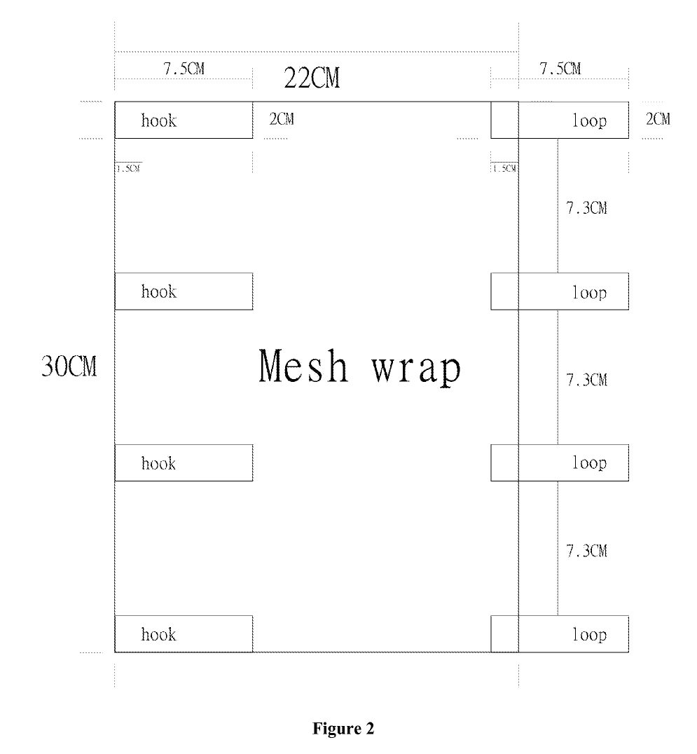

The support body preferably comprises attachment means to provide further support during attachment to the mammal's extremity. In one example, the support body comprises one or more hook and loop structures, wherein one or more hook can be attached to one or more loops on the support body when the cooling sheet is wrapped around the mammal's extremity, thereby helping to further secure the support body (in addition to adhesion to the extremity). An exemplary embodiment of such attachment means is provided below in FIG. 2.

The cooling sheet of the present invention is applied by placing the surface of the adhesive layer in contact with a mammal's extremity, such as a horse's lower leg, at as appropriate (for example, when the mammal's leg has heated up after training or a race). Ordinarily, it is best to cut the sheet to a size sufficient to cover the extremity, for example (for a horse's leg) between about 20-40 cm in length and about 14-30 cm wide. Immediately after application the affected area is rapidly cooled by heat exchange using only the thermal capacity held by the water in the adhesive layer and the support. Since the water retained in the support evaporates easier than the water in the adhesive layer, the water in the support evaporates to cool the lower leg, and then the water in the adhesive layer gradually evaporates to cool the leg continuously for a period of approximately six to eight hours. When needed, water may be sprayed on at regular intervals from the support side to increase the cooling efficiency.

The cooling sheet of the present invention is easy to transport, store and otherwise handle since the surface of the adhesive layer is covered by a water impermeable film. This water impermeable film, for example, is preferably a film prepared from polyester, polyethylene, polyethylene terephthalate, polypropylene, polyvinylidene chloride, polyamide or other materials. In addition, these films may use aluminum foil laminates, may be vapor deposited with aluminum, or may use a film that has a silicon film laminated on the film surface. The means therefor may easily adopt the appropriate means also used in adhesive skin patches for humans and other patches.

### EXAMPLES

Table 1 provides an exemplary formulation for a cooling sheet according to the invention.

TABLE 1

| Ingredient | CAS No. | Percent (%) |
|---|---|---|
| Polyhydric alcohol (glycerin) | 56-81-5 | 15.0 |
| Polyacrylic acid salt | 9003-04-7 | 4.0 |
| Carbopol Ultrez 10 | Proprietary | 2.0 |
| Fine particle size anhydrated silica | 7631-86-9 | 2.5 |
| Aluminum salt | 21645-51-2 | 0.15 |
| Tartaric acid | 87-69-4 | 0.5 |
| Vaseline oil | 8012-95-1 | 2.0 |
| Imidurea | 39236-46-9 | 0.3 |
| RO Water | 7732-18-5 | 73.55 |
| TOTAL | | 100.00 |

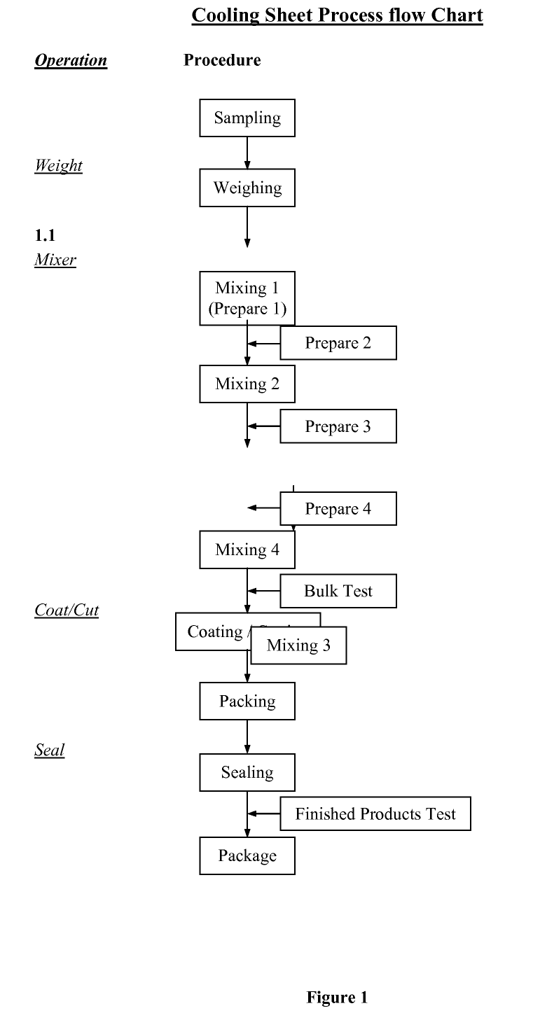

FIG. 1 provides an exemplary flow chart describing a method for preparing a cooling sheet according to the present invention. Table 2 provides a guide to the terms used in the FIG. 1 flow chart.

TABLE 1
Guide to FIG. 1 flow chart

| Operationl Procedure | | Trade name |
|---|---|---|
| Mixing 1 | Prepare 1 | RO Water |
| | | Imidurea |
| | | Carbopol Ultrez 10 |
| Mixing 2 | Prepare 2 | Glycerin (polyhydric alcohol) |
| | | Aluminum hydroxide |
| | | Polyacrylic acid salt |
| Mixing 3 | Prepare 3 | RO Water |
| | | Silicon dioxide |
| | | Tartaric acid |
| Mixing 4 | — | Vaseline oil |

### Evaluation of the Cooling Efficacy of a Second Generation “Extend-A-Cool” Transdermal Cooling System

This study was designed as a proof of concept study to determine the efficacy of a newly designed second generation Extend-A-Cool (EAC) transdermal cooling system when applied to the equine leg. Specifically, the study was conducted to evaluate the reduction of skin surface temperature exhibited by horses fitted with an EAC wrap as per the formulation in Table 1. Upon initial application, there was an immediate reduction of temperature between the leg (skin) and the EAC wrap. The average decrease in temperature between the EAC wrapped leg and the unwrapped leg was 8.0° F. during the seven (7) hours of the recording. This finding was a significant improvement over the reported approximately 6.0° F. decrease in temperature for the cooling sheet disclosed in WO01/08473. Overall the tested version of the EAC product performed very well.

### Materials and Methods

Housing: The test animals were maintained in an area associated with an equine research facility during the study. During the testing period (application of EAC and temperature measurement) the horses were housed in box stalls and observed to prevent interference with the EAC wrap or temperature recording equipment. Water and hay were available ad libitum to the test animals during testing.

Management: The test animals were managed according to normal procedures employed by the test site during the course of data collection.

### Study Design and Treatment Group Assignment:

Treatment group: There were two horses assigned to the study. There was a single treatment group with each of the horses serving as their own control. One leg (right) received the Extend-A-Cool sheet and leg wrap and the opposite (left) leg received only the Extend-A-Cool mesh wrap (no cooling sheet). Temperature sensors were applied to the leg between the skin surface and Extend-A-Cool sheet and wrap (right leg) or the Extend-A-Cool mesh wrap only (left leg).

Temperature recording of the EAC wrapped and unwrapped leg was measured within 20 minutes of the time of placement of the Extend-A-Cool product or mesh wraps (baseline normal temperature), at the time of placement of the treatments (Time 0), and every fifteen (15) minutes for seven (7) hours. The protocol indicated a minimum initial recording time of six hours but this time was extended during the study to seven hours to provide additional data.

Feeding program: Feed and water were provided as per normal study site procedures.

Observations and calculation of variables: Temperature of the legs was measured at the time of placement of the Extend-A-Cool product or mesh wraps (Time 0), and every fifteen (15) minutes for seven (7) hours.

Horses were observed during the course of the study for any unusual or unexpected reaction to the application of the Extend-A-Cool product. There were no abnormal reactions to the product and the both the EAC wrap and mesh over wrap were well tolerated by both horses.

Ambient temperature and humidity of housing/data collection area were recorded during the data collection phase of the study. The building was maintained in a range of temperature of 74 to 77° F. and humidity of 49 to 61%.

### Data Analysis and Summary of Results:

Data were summarized and plotted to graphically depict differences between the control (mesh over-wrap only) legs and the legs wrapped with the new Extend-A-Cool product.

Overall the tested version of the EAC product performed very well. The temperature between the leg (skin) and the EAC wrap began to drop immediately upon application of the EAC wrap. The average decrease in temperature between the EAC wrapped leg and unwrapped leg was 8.0° F. during the seven (7) hours of the recording. This finding was an improvement over the reported approximately 6.0° F. decrease in temperature for the pioneer version of the EAC product.

The product was easy to apply to the legs of the horses and performed as expected. The horses tolerated the product well and did not attempt to rub, chew, or otherwise try to remove from the leg. During the temperature collection times, the horses were restrained by use of a halter and lead rope. Between temperature recordings, the animals were allowed to move freely in box stalls. The EAC product and mesh wraps stayed in place on the legs during the seven hour collection period.

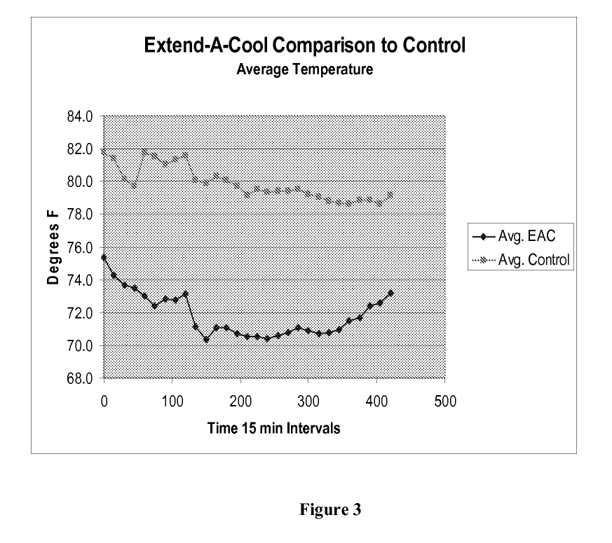

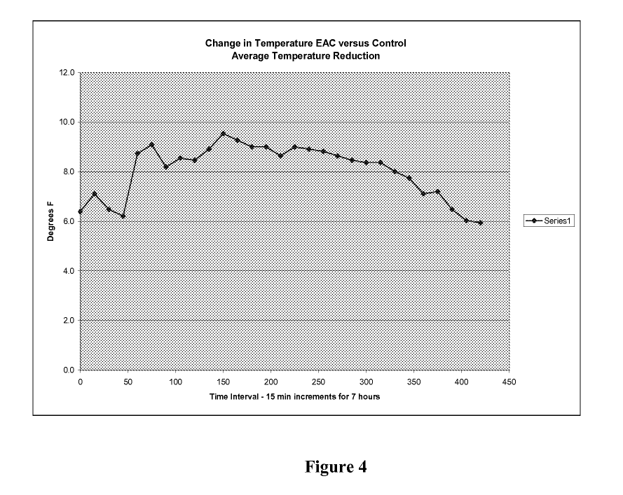

Summarization of the temperature measurement is included in Table 3. Graphic depiction of the temperature comparisons, of the EAC wrap versus the mesh over wrap, over time is presented in FIG. 3. The difference in temperature between the EAC wrap and control is depicted in FIG. 4.

TABLE 3
Summary of Data
Study XX002-06XCxxxx Extend-A-Cool Rutgers Study

| | | | | | Degrees C | | Degrees F | | | | Room |
|---|---|---|---|---|---|---|---|---|---|---|---|
| Time | Temp ° C. Horse #76 | | Temp ° C. Horse #80 | | Avg. | Avg. | Avg. | Avg. | Change | Room Temp | Humidity |
| (min) | Right (EAC) | Left (Control) | Right (EAC) | Left (Control) | EAC | Control | EAC | Control | ° F. | Degrees F | % |
| 0 | 24.6 | 27.6 | 23.6 | 27.7 | 24.1 | 27.7 | 75.4 | 81.8 | 6.4 | 75 | 61 |
| 15 | 23.9 | 27.4 | 23.1 | 27.5 | 23.5 | 27.5 | 74.3 | 81.4 | 7.1 | 75 | 60 |
| 30 | 23.4 | 26.0 | 22.9 | 27.5 | 23.2 | 26.8 | 73.7 | 80.2 | 6.5 | 75 | 59 |
| 45 | 23.6 | 25.7 | 22.5 | 27.3 | 23.1 | 26.5 | 73.5 | 79.7 | 6.2 | 76 | 59 |
| 60 | 23.3 | 28.2 | 22.3 | 27.1 | 22.8 | 27.7 | 73.0 | 81.8 | 8.7 | 76 | 57 |
| 75 | 23.0 | 27.9 | 21.9 | 27.1 | 22.5 | 27.5 | 72.4 | 81.5 | 9.1 | 76 | 56 |
| 90 | 23.3 | 27.5 | 22.1 | 27.0 | 22.7 | 27.3 | 72.9 | 81.1 | 8.2 | 76 | 57 |
| 105 | 23.2 | 27.7 | 22.1 | 27.1 | 22.7 | 27.4 | 72.8 | 81.3 | 8.6 | 76 | 57 |
| 120 | 23.4 | 27.9 | 22.3 | 27.2 | 22.9 | 27.6 | 73.1 | 81.6 | 8.5 | 77 | 57 |
| 135 | 21.8 | 26.3 | 21.7 | 27.1 | 21.8 | 26.7 | 71.2 | 80.1 | 8.9 | 76 | 51 |
| 150 | 21.9 | 27.1 | 20.7 | 26.1 | 21.3 | 26.6 | 70.3 | 79.9 | 9.5 | 76 | 50 |
| 165 | 22.1 | 27.0 | 21.3 | 26.7 | 21.7 | 26.9 | 71.1 | 80.3 | 9.3 | 77 | 52 |
| 180 | 22.1 | 26.9 | 21.3 | 26.5 | 21.7 | 26.7 | 71.1 | 80.1 | 9.0 | 76 | 53 |
| 195 | 21.8 | 26.5 | 21.2 | 26.5 | 21.5 | 26.5 | 70.7 | 79.7 | 9.0 | 75 | 53 |
| 210 | 21.7 | 26.3 | 21.1 | 26.1 | 21.4 | 26.2 | 70.5 | 79.2 | 8.6 | 75 | 52 |
| 225 | 21.9 | 26.6 | 20.9 | 26.2 | 21.4 | 26.4 | 70.5 | 79.5 | 9.0 | 75 | 52 |
| 240 | 21.8 | 26.4 | 20.9 | 26.2 | 21.4 | 26.3 | 70.4 | 79.3 | 8.9 | 76 | 51 |
| 255 | 21.9 | 26.4 | 21.0 | 26.3 | 21.5 | 26.4 | 70.6 | 79.4 | 8.8 | 75 | 51 |
| 270 | 21.9 | 26.4 | 21.2 | 26.3 | 21.6 | 26.4 | 70.8 | 79.4 | 8.6 | 75 | 51 |
| 285 | 22.2 | 26.6 | 21.2 | 26.2 | 21.7 | 26.4 | 71.1 | 79.5 | 8.5 | 76 | 51 |
| 300 | 21.9 | 26.1 | 21.3 | 26.4 | 21.6 | 26.3 | 70.9 | 79.3 | 8.4 | 75 | 49 |
| 315 | 21.9 | 26.0 | 21.1 | 26.3 | 21.5 | 26.2 | 70.7 | 79.1 | 8.4 | 75 | 49 |
| 330 | 21.9 | 25.9 | 21.2 | 26.1 | 21.6 | 26.0 | 70.8 | 78.8 | 8.0 | 75 | 50 |
| 345 | 21.9 | 25.7 | 21.4 | 26.2 | 21.7 | 26.0 | 71.0 | 78.7 | 7.7 | 75 | 50 |
| 360 | 22.4 | 25.9 | 21.5 | 25.9 | 22.0 | 25.9 | 71.5 | 78.6 | 7.1 | 75 | 50 |
| 375 | 22.2 | 25.9 | 21.9 | 26.2 | 22.1 | 26.1 | 71.7 | 78.9 | 7.2 | 75 | 49 |
| 390 | 22.8 | 26.0 | 22.1 | 26.1 | 22.5 | 26.1 | 72.4 | 78.9 | 6.5 | 75 | 50 |
| 405 | 22.8 | 25.8 | 22.3 | 26.0 | 22.6 | 25.9 | 72.6 | 78.6 | 6.0 | 74 | 49 |
| 420 | 23.3 | 26.4 | 22.5 | 26.0 | 22.9 | 26.2 | 73.2 | 79.2 | 5.9 | 74 | 50 |

| | |
|---|---|
| Average | 8.0 |
| ° F. | |
| Change | |

1. A cooling sheet for application to an extremity of a mammal, comprising:
(a) a support body; and
(b) an adhesive layer provided on said support comprising, by weight:
(i) 60-85% of water;
(ii) 5-15% of polyhydric alcohol;
(iii) 0.5%-3% of a crosslinked, acrylic acid-based polymer; and
(iv) 0.1-3% of a compound selected from the group consisting of 5-chloro-2-methyl-4-isothiazolin-3-one; 2-methyl-4-isothiazolin-3-one; 1,2-dibromo-2,4-dicyanobutan; 2-phenoxyethanol and imidurea, or a combination thereof.

2. The cooling sheet of claim 1 wherein the compound comprises imidurea.

3. The cooling sheet of claim 1, wherein the polyhydric alcohol is selected from the group consisting of glycerin, propylene glycol, 1,3-butylene glycol, 1,3-Butanediol, ethylene glycol, and dipropylene glycol, or combinations thereof.

4. The cooling sheet of claim 1, wherein the polyhydric alcohol is 3-10% by weight of glycerin.

5. The cooling sheet of claim 1 further comprising 3-8% polyacrylic acid by weight, or salt thereof.

6. The cooling sheet of claim 5, wherein the polyacrylic acid or salt thereof is selected from the group consisting of sodium polyacrylate and/or a polyacrylate neutralizer, or salts thereof.

7. The cooling sheet of claim 1 further comprising 1-5% oil based ingredient by weight.

8. The cooling sheet of claim 7, wherein the oil-based ingredient is selected from the group consisting of light liquid paraffin, mineral oil, squalane, liquid polyisobutylene, vegetable oils, diisopropyl apidate, isopropyl palmitate, and isopropyl myristate.

9. The cooling sheet of claim 1 further comprising 0.5%-4% anhydrous silica by weight.

10. The cooling sheet of claim 1 further comprising 0.03-0.15% polyvalent metal salt by weight.

11. The cooling sheet of claim 10, wherein the polyvalent metal salt is selected from the group consisting of dried aluminum hydroxide gel, aluminum hydroxide, aluminum sulfate, aluminum acetate, aluminum chloride, aluminum glycinal, alum; and salts of zinc, calcium, copper, lead, iron, tin; and combinations thereof.

12. The cooling sheet of claim 1 further comprising 0.5%-1.5% oxy acid by weight.

13. The cooling sheet of claim 1 further comprising an antiseptic.

14. The cooling sheet of claim 1 further comprising a surfactant

15. The cooling sheet of claim 1, wherein the cooling sheet is adapted to attach to a lower leg portion of a mammal.

16. The cooling sheet of claim 1, wherein the thickness of the adhesive layer is between about 0.8-2.5 mm.

17. The cooling sheet of claim 1, further comprising an impermeable film covering the surface of the adhesive layer.

18. The cooling sheet of claim 1, wherein the support body comprises means for attachment of the support body to the extremity.

19. The cooling sheet of claim 1, wherein the support body comprises one or more hook and loop structures, wherein one or more hook can be attached to one or more loops on the support body when the cooling sheet is wrapped around the mammal's extremity.

20. A method for cooling an extremity of a mammal, comprising placing the surface of the cooling sheet adhesive layer of claim 1 in contact with a mammal's extremity.

21. The method of claim 21, further comprising cutting the cooling sheet to a size sufficient to cover the extremity.

22. The method of claim 20, further comprising spraying water on the cooling sheet.

23. A method for making a cooling sheet, comprising
(a) preparing an adhesive layer comprising:
(i) 60-85% by weight of water;
(ii) 5-15% by weight of polyhydric alcohol;
(iii) 0.5%-3% by weight of a crosslinked, acrylic acid-based polymer; and
(iv) 0.1-3% of a compound selected from the group consisting of 5-chloro-2-methyl-4-isothiazolin-3-one; 2-methyl-4-isothiazolin-3-one; 1,2-dibromo-2,4-dicyanobutan; 2-phenoxyethanol and imidurea, or a combination thereof;
(b) applying the adhesive layer to a support body.

24. The method of claim 23, wherein applying the adhesive layer to a support body comprises applying a kneaded adhesive plaster at an even thickness on one side of the support.

25. The method of claim 23, further comprising forming a water impermeable film on top of the adhesive layer.

26. The method of claim 23, wherein applying the adhesive layer to a support body comprises covering a water impermeable film the adhesive layer, and then forming a support on top thereof.

27. A method for treating periostitis and/or tendonitis in a mammal, comprising contacting a mammal in need thereof with the cooling sheet of claim 1, wherein the contacting is done at an extremity in which the mammal suffers from periostitis and/or tendonitis.

28. The method of claim 27, further comprising cutting the cooling sheet to a size sufficient to cover the extremity.

29. The method of claim 27, further comprising spraying water on the cooling sheet.
